(19) United States
(12) Patent Application Publication
(10) Pub. No.: US 2016/0303265 A1
(43) Pub. Date: Oct. 20, 2016

(54) UV Disinfection System For Ultrasound Probes

(71) Applicant: CIVCO Medical Systems Co., Inc., Kalona, IA (US)

(72) Inventor: Philip Coles, Deep River, CT (US)

(21) Appl. No.: 14/691,234

(22) Filed: Apr. 20, 2015

(51) Int. Cl. A61L 2/10

(52) U.S. Cl. CPC A61L 2/10

(57) ABSTRACT

A disinfection system for intracavity ultrasound probes includes a housing, a disinfection chamber positioned in the housing and receiving at least one ultrasound probe, at least one source of UV light positioned in the chamber, and a vertical drawer slidably positioned in the housing, the vertical drawer having a suspension bracket that accommodates the at least one ultrasound probe such that the probe is suspended in a substantially vertical position. A method for disinfecting intracavity ultrasound probes includes the steps of placing at least one ultrasound probe into a vertical sliding drawer, wherein the probe is received into a suspension bracket positioned in the drawer such that the probe is suspended in a substantially vertical position, sliding the drawer into a disinfection chamber contained within a housing, and supplying UV light via at least one source of UV light positioned in the disinfection chamber to disinfect the probe.

### FIELD OF THE INVENTION

This invention relates generally to disinfection systems for medical devices. More specifically, the present invention relates to disinfection systems for intracavity ultrasound probes using UV light.

### BACKGROUND OF THE INVENTION

All medical instruments that come in contact with mucus membranes or bodily fluids, such as blood, during medical procedures must be carefully disinfected to prevent harmful contamination. There are several types of disinfectants that are used to high-level disinfect or sterilize the medical instruments.

One of the common sterilization techniques used for medical devices is steam sterilization or autoclaving. This technique sterilizes medical equipment by subjecting it to high pressure steam at 121° C. or more, typically for 15 to 20 minutes depending on the size of the medical device. Typically, autoclave system includes a vacuum pump that mechanically removes the air in the sterilizer, allowing it to be more quickly replaced with saturated steam. When the steam has displaced the air, the temperature and steam pressure build until the operating temperature is reached. This operating temperature, the temperature at which sterilization occurs, is maintained for the remainder of the cycle time.

However, autoclave sterilization systems are not suitable for disinfection of heat sensitive medical devices because such devices can be destroyed or have their useful lives severely curtailed by the high temperature and pressures associated with the steam autoclave. The heat sensitive medical devices, therefore, are commonly disinfected using liquid high level disinfections rather than the cheaper and efficient method of steam autoclaving. The two main categories of such heat sensitive instruments are endoscopes and intracavity ultrasound probes.

Endoscopes are typically disinfected in automated washing machines that disinfect and rinse the endoscopes. There are several known automated washing machines marketed by different companies.

Intracavity ultrasound probes, such as vaginal and rectal probes, are examples of heat sensitive medical instruments that cannot be steam autoclaved. The current state of the art in disinfecting such probes is to manually place an ultrasound probe into a container filled with a high level disinfectant for a certain period of time, usually specified by a manufacturer of the disinfection. This is then followed by several fresh water rinses to remove the high level disinfection residue from the probe.

Another method of disinfecting ultrasound probes is by placing a probe into a chamber that is pressurized and flooded with a nebulized hydrogen peroxide. This system is specifically designed for vaginal and rectal ultrasound probes. One example of such system is the system manufactured by Nanosonics Ltd. under the brand Trophon®.

Another prior art system for disinfecting transesophageal (TEE) ultrasound probes utilizes a single use liquid high-level disinfectant (HLD). The TEE probe is placed into a tube that is flooded with the disinfectant after a certain amount of time the HLD is pumped to drain. This is followed by multiple rinses with fresh water. This semi-automated system is manufactured by CS Medical LLC under the brand TD100®.

The intracavity ultrasound probes can also be sterilized by using UV light. There are several prior art systems that utilize UV radiation to disinfect various types of ultrasound probes. In these systems, the probe is typically inserted through an opening in the top of the device and is positioned in a disinfecting chamber having one or more sources of UV radiation.

While these prior art systems have many advantages, there is still a need for improved UV light disinfecting systems.

### SUMMARY OF THE INVENTION

In order to overcome the deficiencies of the prior art, the invention comprises a disinfection system for intracavity ultrasound probes, including a housing, a disinfection chamber positioned in the housing and receiving at least one ultrasound probe, at least one source of UV light positioned in the chamber, a vertical drawer slidably positioned in the housing, the vertical drawer having a suspension bracket that accommodates the at least one ultrasound probe such that the probe is suspended in a substantially vertical position.

In some embodiments, the system further includes at least one sensor positioned inside the disinfection chamber for measuring a quantity of UV light delivered to the chamber. In certain of these embodiments, the system further includes a controller that controls the supply of UV light to the disinfection chamber at least partially based on the calculated quantity. In additional embodiments, the controller includes a processor for recording and storing ultrasound probe profile data for later retrieval by a user.

In certain embodiments, the least one source of UV light comprises four UV lamps positioned around a perimeter of the disinfection chamber. In some of these embodiments, the UV lamps rotate around the at least one ultrasound probe positioned in the disinfection chamber during a disinfection cycle.

In some cases, the disinfection chamber has a reflective inner surface.

In certain embodiments, the system further includes a ventilation system including one or more ventilation openings in the housing and an air circulation fan for cooling off the at least one source of UV light and/or drying off the at least one ultrasound probe positioned in the disinfection chamber.

In some embodiments, the system also includes at least one pair of support members positioned in the disinfection chamber such that the at least one probe is placed between the support members when inserted into the chamber.

In certain embodiments, the system further includes an information acquisition device coupled to the housing for acquiring information about the at least one ultrasound probe being placed in the disinfection chamber. In some of these embodiments, the information acquisition device is a bar code reader. In additional embodiments, the information acquisition device is a radio-frequency identification reader.

In some embodiments, the ultrasound probe comprises at least two probes of different sizes. In certain of these embodiments, the disinfection chamber accommodates the at least two probes of different sizes.

A disinfection system for intracavity ultrasound probes is also provided, including a housing, a disinfection chamber positioned in the housing and receiving at least one ultrasound probe, at least one source of UV light positioned in the chamber, a horizontal drawer slidably positioned in the housing, the horizontal drawer having at least two adjustable brackets that accommodate the at least one ultrasound probe such that the probe is positioned in a substantially horizontal position, wherein the at least one adjustable brackets are movable with respect to each other to accommodate different types of ultrasound probes.

A method for disinfecting intracavity ultrasound probes is further provided, including the steps of placing at least one ultrasound probe into a vertical sliding drawer, wherein the probe is received into a suspension bracket positioned in the drawer such that the probe is suspended in a substantially vertical position, sliding the drawer into a disinfection chamber contained within a housing, and supplying UV light via at least one source of UV light positioned in the disinfection chamber to disinfect the probe.

In some embodiments, the sliding drawer is opened and closed manually. In additional embodiments, the sliding drawer is opened and closed automatically.

In certain embodiments, the method further includes the step of acquiring information about the at least one ultrasound probe via an information acquisition device prior to positioning the probe into the sliding drawer. In some of these embodiments, the method further includes the step of acquiring information about the at least one ultrasound probe via the information acquisition device after the disinfection is completed and the probe is withdrawn from the sliding drawer.

In some embodiments, the method also includes the steps of measuring a quantity of UV light delivered to the disinfection chamber via at least one sensor positioned in the chamber and controlling the supply of UV light to the disinfection chamber via a controller based at least partially on the calculated quantity. In certain of these embodiments, the method further includes the step of recording and storing ultrasound probe profile data via the controller for later retrieval by a user.

In certain embodiments, the method also includes the steps of supplying air to the disinfection chamber via at least one opening provided in the housing and circulating the air within the chamber via an air circulation fan to cool off the at least one source of UV light and/or dry off the at least one ultrasound probe positioned in the disinfection chamber.

In some cases, the method also includes the step of rinsing the at least one ultrasound probe positioned in the disinfection chamber with a disinfecting solution prior to the step of supplying UV light to the chamber via the at least one source of UV light.

In certain embodiments, the at least one source of UV light comprises two or more UV lamps and the method further includes the step of rotating the lamps around the at least one ultrasound probe positioned in the disinfection chamber.

Other objects of the invention and its particular features and advantages will become more apparent from consideration of the following drawings and accompanying detailed description.

### BRIEF DESCRIPTION OF THE DRAWINGS

FIG. 1A is a front perspective view of the disinfecting system in accordance with the present invention.

FIG. 1B is a back perspective view of the disinfecting system of FIG. 1A, with two ultrasound probes.

FIG. 2A is a cross-sectional view of the disinfecting system of FIG. 1A, taken along the line “2-2.”

FIG. 2B is another cross-sectional view of the disinfecting system of FIG. 1A, taken along the line “2-2.”

FIG. 3 is a cross-sectional view of the disinfecting system of FIG. 1A, taken along the line “3-3.”

FIG. 4 is a cross-sectional view of the disinfecting system of FIG. 1A, taken along the line “4-4.”

FIG. 5A is a perspective view of the disinfecting system of the present invention with a horizontal drawer.

FIG. 5B is a perspective view of the disinfecting system of FIG. 5A, with the drawer opened.

FIG. 6 is a cross-sectional view of the disinfecting system of FIG. 5A, taken along the line “6-6.”

### DETAILED DESCRIPTION OF THE INVENTION

The present teachings are described more fully hereinafter with reference to the accompanying drawings, in which the present embodiments are shown. The following description is presented for illustrative purposes only and the present teachings should not be limited to these embodiments.

As used in the description, the terms “top,” “bottom,” “above,” “below,” “over,” “under,” “above,” “beneath,” “on top,” “underneath,” “up,” “down,” “upper,” “lower,” “front,” “rear,” “back,” “forward” and “backward” refer to the objects referenced when in the orientation illustrated in the drawings, which orientation is not necessary for achieving the objects of the invention.

The disinfection system of the present invention is particularly suitable for disinfecting vaginal/rectal ultrasound probes. However, it should be understood that other kinds of intracavity probes, such as transesophageal ultrasound probes, or other type of ultrasound probes may be disinfected using the system and method of the present invention.

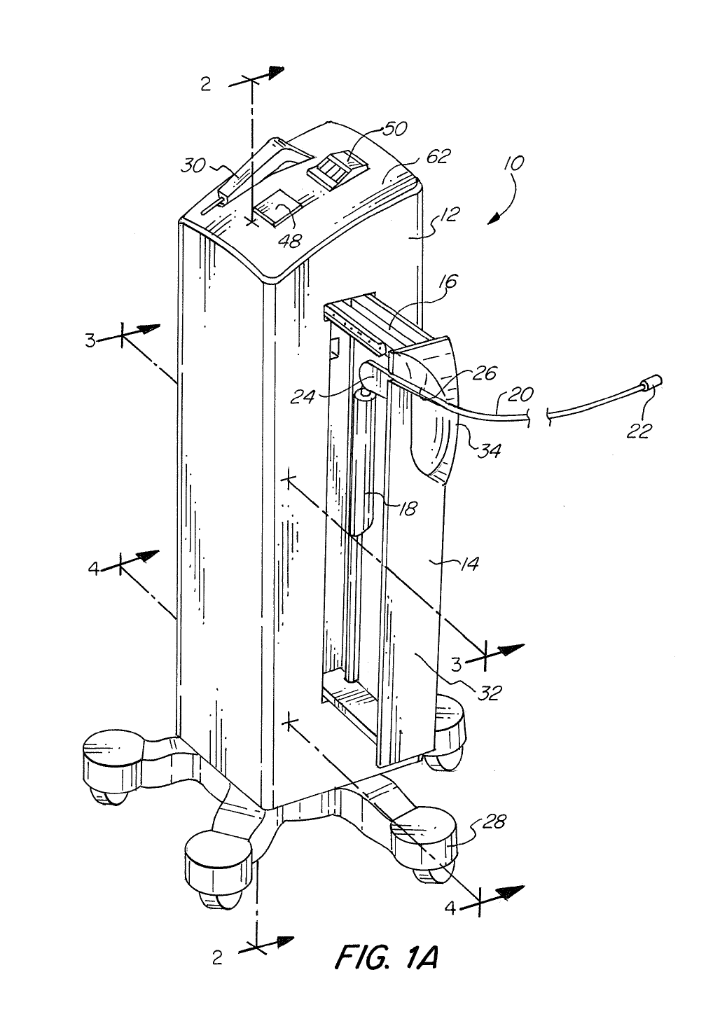

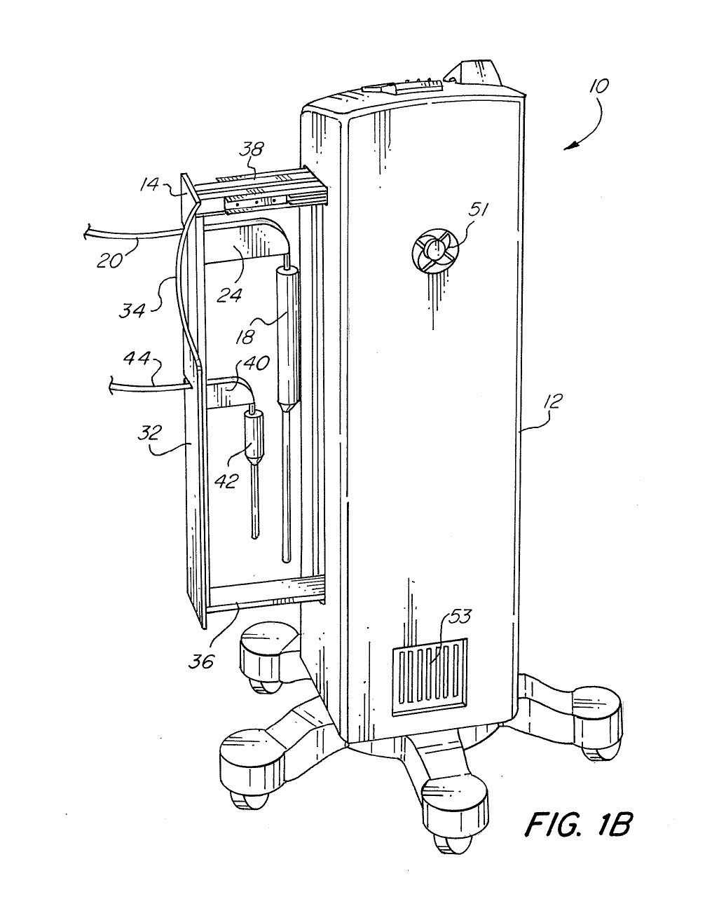

One exemplary embodiment of the disinfection system of the present invention is illustrated in FIGS. 1A and 1B. As shown in this figure, the disinfection system (10) has a housing (12) that houses all components of the system (10). The housing has any suitable shape that is capable of accommodating an ultrasound probe and is made with any suitable material, such as, e.g., hard plastic, etc.

In the embodiment shown in FIGS. 1A and 1B, the housing (12) is a standalone unit which can be placed on the floor anywhere in a physician's office or a hospital facility, e.g., an operating room. In this embodiment, the housing (12) includes a plurality of legs with wheels (28) positioned on the bottom of the housing that allow the housing (12) to be easily moved around to a desired location, e.g. next to ultrasound imaging unit or next to a sink for discarding disinfecting byproducts. It is understood, however, that in other embodiments, the housing may be placed on any flat surface, such as floor or table, and not have any wheels. In additional embodiments, the housing (12) may be permanently attached to a surface, such as a wall or a table.

The housing (12) has a vertical sliding drawer (14) that slides out of one of the side walls of the housing. As more clearly shown in FIG. 1B, the sliding drawer (14) has a front wall (32), a top wall (38), a bottom wall (36), and a back wall. The inside of the sliding drawer between the front, top, back and bottom walls is hollow for accommodating at least one ultrasound probe therein. The size of the housing (12) and/or the sliding drawer (14) depends on the type of ultrasound probe being disinfected. For example, vaginal/rectal ultrasound probes are typically about 40 cm long, and the transesophageal ultrasound probes are typically about 1.3 meters long. Accordingly, the size of the housing and/or drawer will be chosen such that it can accommodate a particular kind of the ultrasound probe.

The sliding drawer also has a sliding mechanism (16) positioned on the top wall (38) and/or the bottom wall (36) that allows the drawer (14) to slide in and out of the housing (12), as described in more detail below. Any suitable sliding mechanism may be used in accordance with the present invention. For example, a pair of corresponding rails—one positioned on the top and/or bottom wall of the sliding drawer and the other positioned in the housing—that cooperate in a sliding arrangement may be used. In other embodiments, the sliding mechanism is a ball and catch type of mechanism. It is understood that any other suitable mechanism may be used. In some advantageous embodiments, the sliding mechanism includes one or more stoppers that prevent the sliding drawer (14) from sliding too far out of the housing (12) and/or sliding too far into the housing. Any suitable stopper may be used.

In the embodiments where the sliding drawer (14) is operated manually, the front wall (32) of the sliding drawer (12) includes a grasping surface (34) for grasping of the drawer by a user. As shown in FIG. 1B, the grasping surface (34) curves outwardly from the front wall of the drawer such that the user's fingers may be inserted into the space behind the grasping surface (34). In other embodiments, the front wall (32) of the housing may have a handle or any other suitable mechanism that allows the user to open and close the sliding drawer.

The sliding drawer (14) includes a suspension bracket (24) positioned adjacent the top wall (38) of the housing (12). The suspension bracket accommodates an ultrasound probe (18), as shown in FIG. 1B. The suspension bracket (24) has a groove on its top portion such that a cable (20) of the ultrasound probe (18) is inserted therein, allowing the ultrasound probe itself to hang downwardly from the suspension bracket in a substantially vertical orientation. In some advantageous embodiments, the groove is lined with a gripping material, such as silicone, to ensure that the cable (20) of the probe (18) is securely held in the suspension bracket (24). The front wall (32) of the sliding drawer (14) has a recess (26) for accommodating the cable (20) such that it extends out of the housing through the recess when the sliding drawer is closed. When in use, the cable (20) is inserted into the sliding drawer (14) through the recess (26) and is positioned in the suspension bracket (24), while the ultrasound probe (18) is suspended from the bracket inside the sliding drawer (14).

It is understood that any other type of suspension mechanism may be used in accordance with the present invention. It is also noted that in additional embodiments, the sliding drawer (14) can include two or more suspension brackets for accommodating two or more ultrasound probes for simultaneous disinfection. For example, as shown in FIG. 1B, the sliding drawer (14) includes a second suspension bracket (40) positioned below the first suspension bracket (24). The second suspension bracket (40) accommodates a cable (44) of a second ultrasound probe (42) that is suspended from the bracket in a substantially vertical position. It is noted that two or more suspension brackets may have any other suitable arrangement inside the sliding drawer (14).

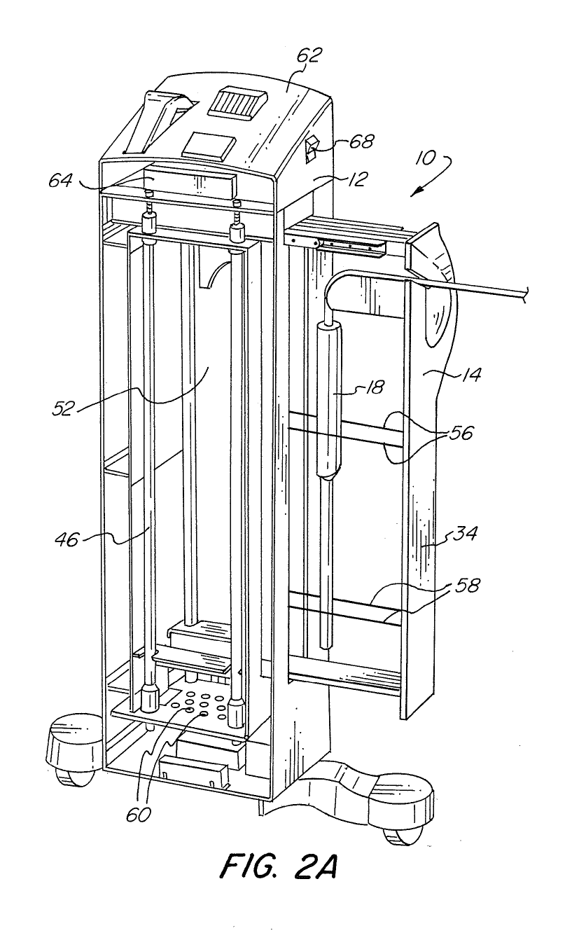

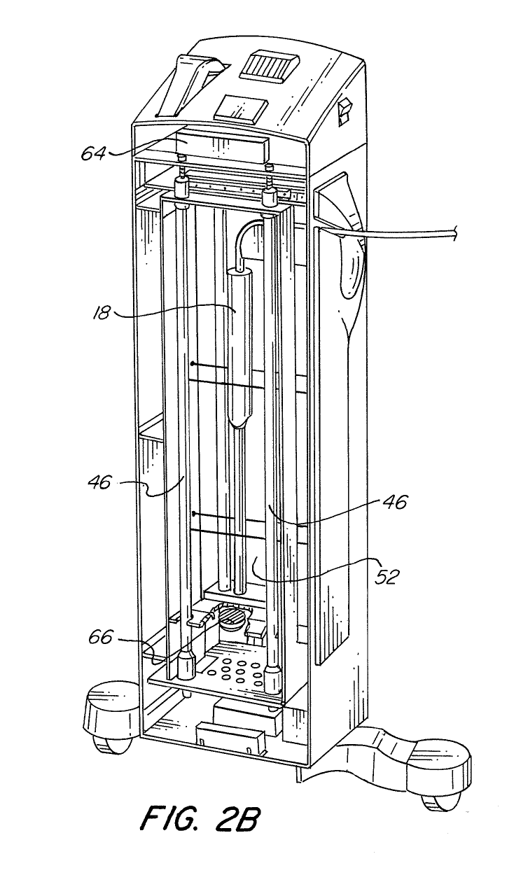

As shown in the cross-sectional views in FIGS. 2A and 2B, the system further includes a disinfection chamber (52) positioned inside the housing (12). The disinfection chamber (52) may have any shape suitable for accommodating one or more ultrasound probes. The sliding drawer (14) slides inside the chamber (52), when in a closed position, as illustrated in FIG. 2B. This way, the ultrasound probe (18) suspended in the sliding drawer (14) is placed inside the disinfection chamber (52) for disinfection.

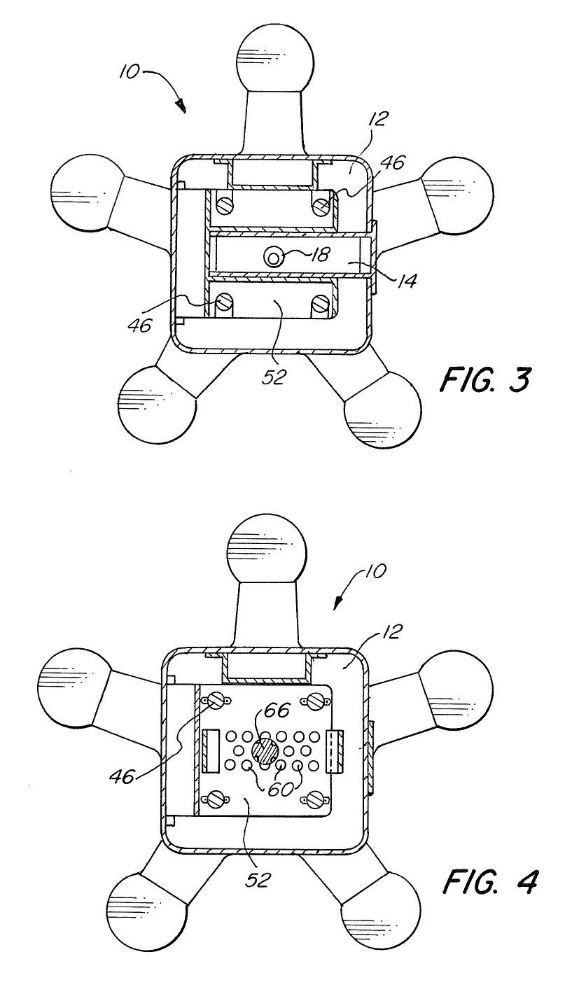

The chamber (52) has one or more sources of UV light positioned therein. In the embodiment shown in FIGS. 2A-4, the chamber (52) includes four UV lamps (46) positioned around the perimeter of the chamber at equal distances from each other. The lamps extend substantially through the entire height of the disinfection chamber (52) and are secured to the top and bottom walls of the chamber. This design ensures that the entire surface of the ultrasound probe (18) placed in the chamber is uniformly exposed to UV radiation. It is understood that any type, number, size and arrangement of UV sources may be used in accordance with the present invention.

In some embodiments, one or more UV sources rotate around the ultrasound probe positioned in the disinfection chamber during the disinfection cycle. This facilitates uniform exposure of the ultrasound probe to UV radiation to ensure proper disinfection. Instead of being secured to the top and bottom walls of the chamber (52), the UV lamps are positioned on one or more rotating platforms placed in the chamber, which rotate the lamps around the probe. Alternatively, the top and bottom walls of the chamber may rotate, with the UV lamps being secured to the walls. Any other suitable rotation mechanism may also be used.

In some advantageous embodiments, the inside surface of the disinfection chamber (52) is lined with reflective material, such as, e.g., polished aluminum, mirrored glass, etc. The reflective material facilitates disinfection by reflecting the UV light delivered by the UV sources (46), such that the UV light is evenly distributed throughout the surface of the ultrasound probe (18) positioned in the chamber (52).

The disinfection chamber (52) also includes one or more sensors that measure a quantity of UV light delivered to the chamber by the UV light sources. The sensors are positioned at any suitable location inside the disinfection chamber (52) such that they are capable measuring the amount of UV light delivered to the ultrasound probe (18) positioned in the chamber. In one exemplary embodiment shown in FIG. 2B, the sensors (66) are positioned on the underside of the bottom wall (36) of the sliding drawer (24). There is space between the bottom wall of the drawer (24) and the bottom of the chamber (52), and the UV lamps (46) extend through the entire length of the chamber (52). The sensors (66) include four sensors, with each sensors oriented towards one of the UV lamps (46), as better shown in FIG. 4. This way, each sensor measures the UV radiation emitted from one of the UV lamps.

The sensors (66) are capable of measuring both the UV light output directly from the UV lamps and the UV light reflected from the inner reflective surface of the disinfection chamber (52) to provide a more accurate measurement of UV radiation delivered to the ultrasound probe. The sensors (66) are also capable of determining if one or more of the UV lamps (46) fails and transmitting that information to the system's CPU such that this issue may be quickly resolved by the user.

In other embodiments, the sensors may be positioned on the top side of the bottom wall of the drawer facing the ultrasound probe. In additional embodiments, the sensors may be positioned on the bottom of the chamber (52). Any suitable type of sensors may be used in accordance with the present invention.

The information from the sensors (66) is continuously transmitted to the system's CPU. The CPU operates the UV light sources and controls the supply of the UV light based at least in part on the measured quantity of the UV light received from the sensors (66).

The top portion (62) of the housing (12) includes various other components of the disinfecting system (10). For example, as shown in FIG. 2A, there is a touch screen display (48) that shows the progress of the disinfection cycle to the user, and also allows for interaction between the user and the system (10). Any suitable touch screen display may be used. The display (48) may include LED lights that visually indicate the progress of the disinfection cycle to the user. The touch screen may display various commands selectable by the user by simply pressing the buttons on the touch screen.

The top portion of the housing also houses a central processing unit (CPU) (64) that controls the operation of the system. Any suitable type of the processor may be used in accordance with the present invention. The CPU (64) is connected to the touch screen display (48) such that is sends the information regarding the disinfection process to the touch screen for display to the user and receives commands from the user via the touch screen. The CPU (64) stores information about a plurality of disinfection cycles such that the information may be later retrieved by the user.

The top portion of the housing further includes a printer (50) that generates printout reports with the information about each disinfecting cycle based on the information received from the CPU (64). A USB port (68) may also be provided for connection to a flash drive memory device. The USB port (68) is used to retrieve information about the previous disinfection cycles stored by the CPU. The system may also include an internal storage device (not shown) for storing information about the disinfection cycles.

The system (10) also includes an information acquisition device (30) for acquiring information about the ultrasound probe being disinfected. Any suitable type of information acquisition device may be used. In some advantageous embodiments, the information acquisition device (30) is a bar code reader that acquires information from a bar code label associated with the ultrasound probe. In other advantageous embodiments, the information acquisition device is a radio-frequency identification reader capable of acquiring information from a radio-frequency identification chip associated with the ultrasound probe.

In the embodiment shown in FIG. 1A, the information acquisition device (30) is placed into a recess on the top portion of the housing when not in use. The information acquisition device (30) is connected to the housing/CPU via a wired or a wireless connection. It is understood that, in other embodiments, the information acquisition device (30) may be a standalone unit separate from the housing (12).

Before the ultrasound probe (18) is positioned into the sliding drawer (14), the information acquisition device (30) is used to scan the bar code or the RFID chip positioned on the ultrasound probe to acquire information associated with the probe, e.g., probe type, last disinfection process, etc. The bar code or the RFID chip may be positioned anywhere on the ultrasound probe (18). In some advantageous embodiments, as shown in FIG. 1A, the bar code or the RFID chip (22) is positioned on the cable connector portion at the proximal end of the ultrasound probe cable (20). The information from the device (30) is sent to the CPU (64).

Then, after the disinfection cycle is completed and the ultrasound probe (18) is withdrawn from the housing (12), the information acquisition device (30) is used again to scan the probe. The device (30) is also capable of acquiring various information associated with the disinfection cycle, such as operator ID, patient ID, amount of radiation emitted during disinfecting cycle, etc. The CPU (64) records and stores the ultrasound probe information acquired by the information acquisition device (30), as well as other information associated with each disinfection cycle, for later retrieval by the user. It is noted, however, that in some embodiments, the ultrasound probe (18) does not need to be scanned at the end of the disinfection cycle.

It is understood that the arrangement of different system components shown in FIG. 2A is only exemplary and that any other arrangement may be used without departing from the spirit of the present invention.

In some advantageous embodiments, the disinfection system of the present invention further includes at least one pair of support members positioned in the disinfection chamber (52). The support members function to keep the ultrasound probe (18) positioned in the chamber (52) in a substantially fixed vertical position during the disinfection cycle. As illustrated in FIG. 2B, the support members are a pair of horizontally oriented wires (54) extending parallel to each inside the chamber (52). The distance between the wires (54) is such that the ultrasound probe (18) can be inserted therebetween when placed in the disinfection chamber (52). The wires are made out of steel or any other suitable material. It is understood that more than one pair of support members may be provided in the chamber. For example, two or three pairs of corresponding wires may be positioned at different heights inside the chamber (52) to help to maintain the probe (18) in a fixed position.

In additional embodiments, similar support members may be provided in the sliding drawer (24). For example, as shown in FIG. 2A, the sliding drawer includes one or more pairs of wires (56, 58) extending horizontally from the front wall to the back wall of the drawer (24). When the ultrasound probe is positioned in the sliding drawer, it is inserted between the corresponding wires such that the probe (18) is maintained is a fixed vertical position inside the drawer.

In some embodiments, the system of the present invention also includes a ventilation system. The ventilation system is used for cooling off the UV light sources (46) and/or for drying off the ultrasound probe (18) being disinfected. The ventilation system includes a plurality of openings in the housing that allow the air inside the disinfection chamber. As shown in FIG. 2A, the plurality of openings (60) are positioned on the bottom wall of the disinfection chamber (52) and are connected to a plurality of openings (53) in the outer wall of the housing. Outside air enters the disinfection chamber (52) through the openings (53, 60) and cools off the UV light sources (46) during the disinfection cycle. The air is also used to dry the ultrasound probe (18), which may be wet from a prewash with a disinfecting solution. It is understood that the openings may be provided at any other suitable location or a plurality of locations in the housing (12).

In additional embodiments, the ventilation system further includes a fan positioned in the housing. Any suitable fan type may be used. As illustrated in FIG. 1B, the fan (51) is positioned in a back wall of the housing (12) towards the top portion of the housing. It is noted that the fan may be placed in any other suitable location in the housing. The fan (51) is connected with the inside of the disinfection chamber (52). When the fan (51) is turned on, it draws air from the openings (53, 60) and circulates it through the disinfecting chamber (52) to dry off the ultrasound probe and/or cool off the UV light sources. The fan (51) may be provided with an air filter, such as a high efficiency particulate air (HEPA) filter, to remove particles from the air prior to it being circulated through the disinfection chamber to facilitate an efficient disinfection process and to prevent contamination of the UV light sources.

The disinfecting chamber (52) may also include at least one temperature sensor positioned in the chamber for measuring the temperature inside the chamber. The measured temperature in then transmitted to the CPU (64), which controls the operation of the fan (51) at least partially based on the measured temperature. This way, the fan is operated by the CPU to cool off the chamber (52) only when the temperature inside the chamber rises above a predetermined threshold level.

However, in other embodiments, the fan (51) is operated continuously when the disinfecting system is in operation to facilitate drying of the ultrasound probe(s). In these embodiments, the temperature sensor may be omitted.

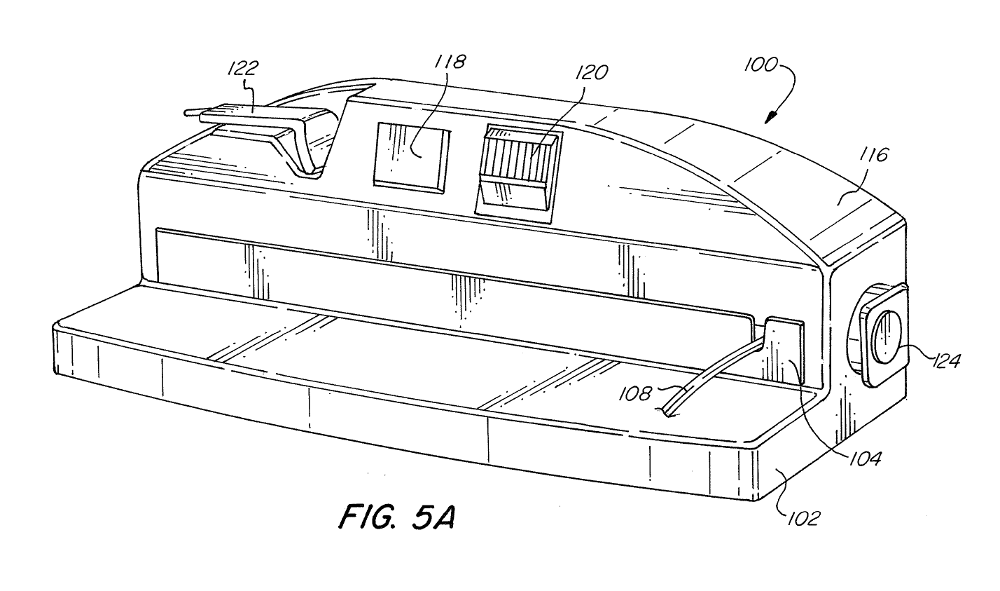

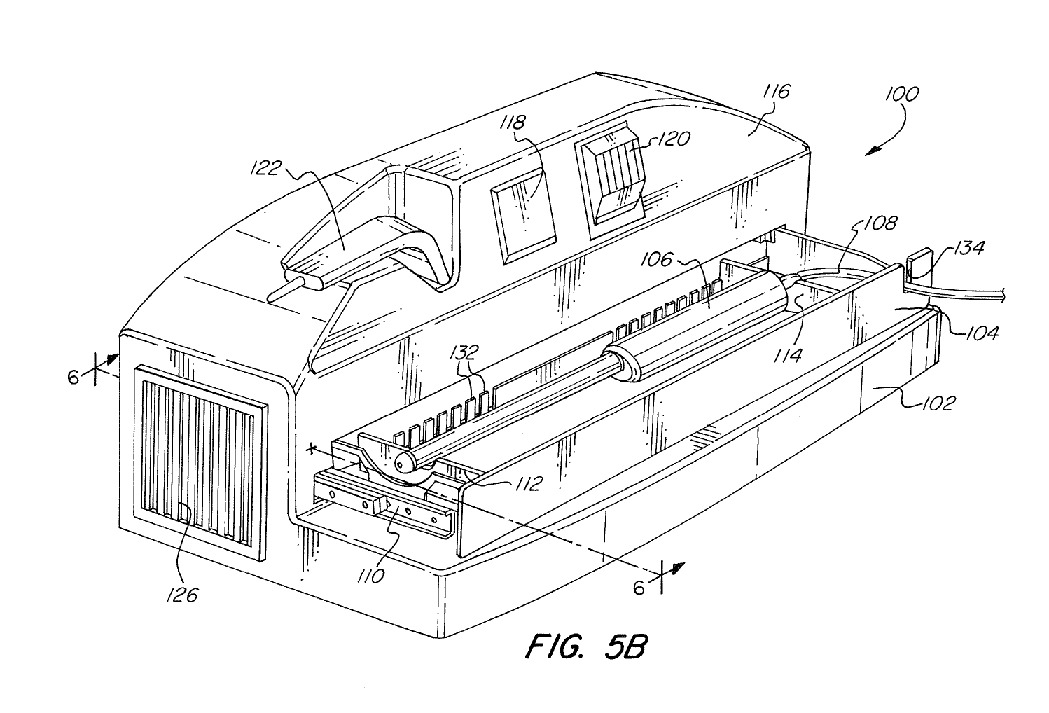

FIGS. 5A and 5B illustrate another exemplary embodiment of the disinfecting system of the present invention. In this embodiment, the disinfecting system (100) has a more compact housing (102) with a bottom wall designed to be placed on any flat surface, such as a cabinet or table top. This way, the unit may be simply placed on any flat surface in the physician's office.

The disinfecting system (100) includes a horizontally oriented sliding drawer (104) that slides in and out of the housing (102). As shown in FIG. 5B, the drawer (104) accommodates at least one ultrasound probe (106). The drawer has at least two support brackets (112) and (114) that receive the ultrasound probe (106) such that the probe is positioned in a substantially horizontal positioned inside the drawer. The brackets may have any suitable shape and may be made with any suitable material. In some advantageous embodiments, the brackets are made with a substantially transparent material that transmits UV light to ensure that all parts of the probe (106) are exposed to UV light during the disinfection cycle.

The drawer (104) has a plurality of spacers (132) that accommodate the support brackets (112, 114), such that the brackets can be moved to a different distance from each other to accommodate different types and sizes of ultrasound probes. A front wall of the sliding drawer (104) has a recess (134) that accommodates a cable (108) of the ultrasound probe (106) once the probe is placed within the drawer. The drawer (104) has any suitable sliding mechanism, as described above with respect to the vertical sliding drawer shown in FIGS. 1A-4.

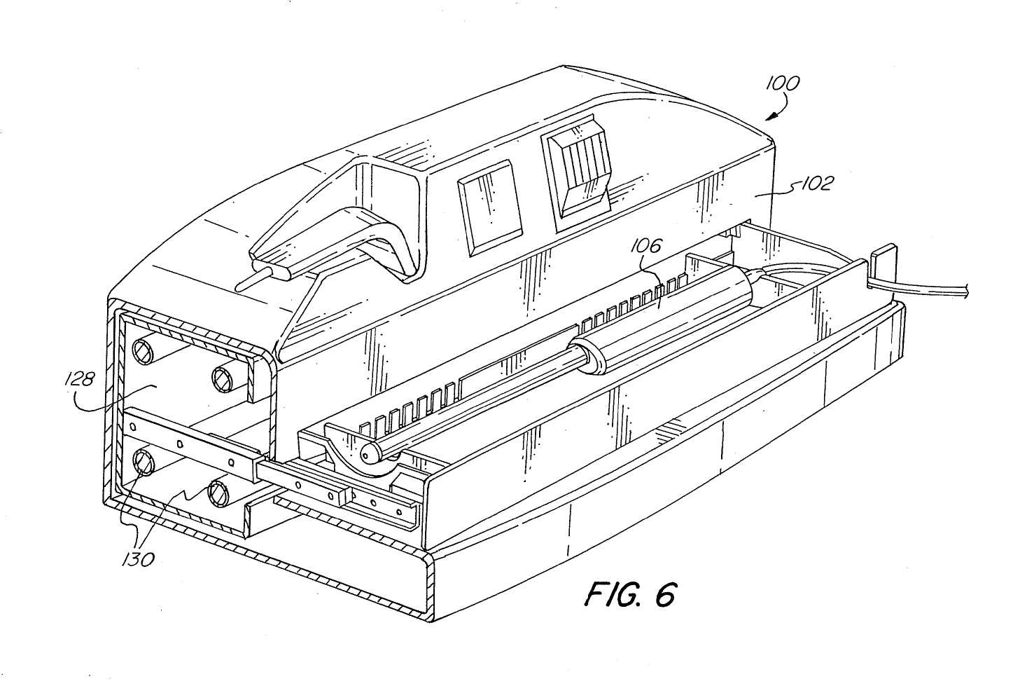

As shown in FIG. 6, the housing (102) includes a disinfection chamber (128), similar to the disinfection chamber (52) described above. The disinfection chamber (128) has a plurality of UV light sources (130) positioned therein. Once the ultrasound probe (106) is placed in the support brackets (112, 114) in the sliding drawer (104), the drawer is slid inside the housing (102), such that the probe (106) is placed within the disinfection chamber (128). Then, the UV light sources (130) are turned on to expose the probe to the UV light.

The housing (102) includes a top portion positioned above the disinfecting chamber (128) that houses other components of the system. As shown in FIGS. 5A and 5B, the system also includes an information acquisition device (122), a touch screen display (118) and a printer (120). The housing further houses a CPU (not shown) that controls the operation of the system. There is also a UBS port (not shown) for transfer of the information about the disinfection cycles to a memory device. The operation of these system components is the same as described above with respect to FIGS. 1A-4.

The disinfecting system (100) also has a ventilation system including a plurality of openings (126) in the housing that let the outside air into the disinfecting chamber (128) and a fan (124) that circulates the air through the chamber (128). As shown in FIGS. 5A and 5B, the openings (126) are positioned on one side wall of the housing (102) and the fan is positioned on the opposite side wall of the housing. The air is circulated through the chamber (102) to cool off the UV light sources (130) and/or to dry off the ultrasound probe (106) positioned in the chamber.

In some embodiments, the disinfecting system of the present invention performs a rinse cycle prior to the UV radiation disinfection cycle to wash away impurities, e.g. gel residue, bodily fluids and/or tissue, from the ultrasound probe. Any suitable enzymatic solution may be used to perform the rinse cycle. The enzymatic solution is supplied to the chamber from a container, may be circulated through the chamber several times and then discarded into the drain or into the same container for reuse.

The following is a description of an exemplary method of operation of the disinfecting system in accordance with one advantageous embodiment of the present invention.

When the system (10) is turned on, the display (48) reads “ready for use/press enter.” Once the operator presses “enter,” the display (48) reads “is probe pre-cleaned?” with a “yes/no” selection. The operator must select “yes” or “no.” If he or she selects “no,” they will not be able to continue.

The information acquisition device (30) is then turned on and the display (48) reads “scan probe connector.” The operator picks up the hand held device (30) and scans a bar code or an RFID chip (22) provided on the electrical connector of the ultrasound probe. This connector may still be attached to the ultrasound machine. An audible beep will confirm that the device (30) has read the bar code/RFID chip, and the device (30) will turn off.

Next, the display (48) reads “open drawer/insert probe.” The sliding drawer (14) is opened manually or automatically, and the operator inserts the ultrasound probe (18) into the drawer by placing the probe cable (20) into the suspension bracket (24) such that the probe is suspended from the bracket. One or more sensors positioned on the bracket (24) detect that probe (18) is installed. The display (28) then reads “close drawer,” and the drawer (14) is closed manually or automatically. An electrically operated interlock prevents the drawer (14) from being open until the end of the disinfecting cycle.

The activation of the interlock turns on the ventilation fan (51) that draws room air through a HEPA filter to provide cooling of the UV light sources (46) in the disinfection chamber (52). The fan (51) will run throughout the disinfection cycle. A warning light will indicate fan failure.

The information acquisition device (30) may be turned on again when the drawer interlock activates, and the display (48) will read “scan operator ID.” Unique bar code cards may be provided for operator ID's or the operators may use their existing employee ID cards. The operator then scans his or her ID card. An audible beep will confirm that the device (30) has read the ID card. The display (48) may also optionally read “scan patient ID,” to prompt the operator to scan the patient ID with the device (30), if desired. The device (30) is then turned off.

Next, the UV light sources (46) turn on and the display (48) reads “disinfection in progress/time remaining—xxx minutes.” The timer starts the disinfection cycle and the time remaining will be displayed as counting down from a pre-determined disinfection cycle time. In some embodiments, the disinfection cycle is five minutes. However, it is understood that any desired disinfection cycle time may be programmed into the system.

At least four UV light sensors (66) are positioned in the disinfection chamber (52) in such a manner as to be able to measure the output from each of the UV lamps (46) at the center of a target area of the ultrasound probe being disinfected, as described above. The sensors have the ability to read the continuous UV light output from each UV lamp for the entire period of disinfection. This information is displayed in real time on the touch screen display (48) in the form of a bar-chart that will show the acceptable level of UV light output necessary to provide for the required log reduction of bacteria. Other methods of displaying this information are also envisaged. In the event that the predetermined level of UV light output is not reached during the required disinfection time, the device will abort the cycle and the display (48) will read “error/probe not disinfected.” This message may also be displayed in the event that any other error is detected, including, but not limited to, a power failure.

At the end of a complete and successful disinfecting cycle, which is determined by the fact that the minimum required output of UV light was detected for the required time, the UV lamps (46) are turned off and the display (48) reads: “cycle complete PASS/remove the probe.”

After the disinfecting cycle is completed, the printer (50) generates two reports. One version will be attached to the log and will contain the following information:

a. Time/Date

b. Pass/Fail

c. Probe ID

d. UV light output

e. Operator ID

f. Patient ID (optional)

The second printout will be attached to a custom tag provided with the disinfecting system that will then be attached to the disinfected ultrasound probe. This label/tag will stay with the probe until the probe is used on the next patient, at which time the label/tag will be removed and placed in the patient file. This will confirm when the probe was last used and that it was successfully disinfected. The second printout will contain the following information:

a. Time/Date

b. Pass/Fail

c. Probe ID

In addition to generating the printouts, the system may include an external USB port (68) to allow for data to be transferred via a flash drive or any other memory device.

The display (48) will also alert the operator when the UV light lamps and/or the HEPA air filter are required to be changed, which is typically annually. A two-week warning period is envisaged. If the lamps and/or the filter are not changed within that period, the device will not start.

It would be appreciated by those skilled in the art that various changes and modifications can be made to the illustrated embodiments without departing from the spirit of the present invention. All such modifications and changes are intended to be covered hereby.

What is claimed is:

1. A disinfection system for intracavity ultrasound probes, comprising:
a housing;
a disinfection chamber positioned in said housing and receiving at least one ultrasound probe;
at least one source of UV light positioned in said chamber; and
a vertical drawer slidably positioned in said housing, said vertical drawer having a suspension bracket that accommodates the at least one ultrasound probe such that the probe is suspended in a substantially vertical position.

2. The disinfection system of claim 1, further comprising at least one sensor positioned inside said disinfection chamber for measuring a quantity of UV light delivered to the chamber.

3. The disinfection system of claim 2, further comprising a controller that controls the supply of UV light to said disinfection chamber at least partially based on the measured quantity.

4. The disinfection system of claim 1, further comprising a processor for recording and storing ultrasound probe profile data and/or information associated with a disinfection cycle for later retrieval by a user.

5. The disinfection system of claim 1, wherein the least one source of UV light comprises four UV lamps positioned around a perimeter of said disinfection chamber.

6. The disinfection system of claim 5, wherein the UV lamps rotate around the at least one ultrasound probe positioned in the disinfection chamber during a disinfection cycle.

7. The disinfection system of claim 1, wherein said disinfection chamber has a reflective inner surface.

8. The disinfection system of claim 1, further comprising a ventilation system including one or more ventilation openings in said housing and an air circulation fan for cooling off the at least one source of UV light and/or drying off the at least one ultrasound probe positioned in the disinfection chamber.

9. The disinfection system of claim 1, further comprising at least one pair of support members positioned in the disinfection chamber such that the at least one probe is placed between the support members when inserted into the chamber.

10. The disinfection system of claim 1, further comprising an information acquisition device coupled to the housing for acquiring information about the at least one ultrasound probe being placed in the disinfection chamber.

11. The disinfection system of claim 10, wherein the information acquisition device comprises a bar code reader.

12. The disinfection system of claim 10, wherein the information acquisition device comprises a radio-frequency identification reader.

13. The disinfection system of claim 1, wherein said ultrasound probe comprises at least two probes of different sizes.

14. The disinfection system of claim 13, wherein said disinfection chamber accommodates the at least two probes of different sizes.

15. A disinfection system for intracavity ultrasound probes, comprising:
a housing;
a disinfection chamber positioned in said housing and receiving at least one ultrasound probe;
at least one source of UV light positioned in said chamber;
a horizontal drawer slidably positioned in said housing, said horizontal drawer having at least two adjustable brackets that accommodate the at least one ultrasound probe such that the probe is positioned in a substantially horizontal position, wherein the at least one adjustable brackets are movable with respect to each other to accommodate different types of ultrasound probes.

16. A method for disinfecting intracavity ultrasound probes, comprising the steps of:
placing at least one ultrasound probe into a vertical sliding drawer, wherein the probe is received into a suspension bracket positioned in the drawer such that the probe is suspended in a substantially vertical position;
sliding the drawer into a disinfection chamber contained within a housing; and
supplying UV light via at least one source of UV light positioned in said disinfection chamber to disinfect the probe.

17. The method of claim 16, wherein the sliding drawer is opened and closed manually.

18. The method of claim 16, wherein the sliding drawer is opened and closed automatically.

19. The method of claim 16, further comprising the step of acquiring information about the at least one ultrasound probe via an information acquisition device prior to positioning the probe into the sliding drawer.

20. The method of claim 19, further comprising the step of acquiring information about the at least one ultrasound probe via the information acquisition device after the disinfection is completed and the probe is withdrawn from the sliding drawer.

21. The method of claim 16, further comprising the steps of measuring a quantity of UV light delivered to the disinfection chamber via at least one sensor positioned in the chamber and controlling the supply of UV light to said disinfection chamber via a controller based at least partially on the measured quantity.

22. The method of claim 21, further comprising the step of recording and storing ultrasound probe profile data via said controller for later retrieval by a user.

23. The method of claim 16, further comprising the steps of supplying air to the disinfection chamber via at least one opening provided in the housing and circulating the air within the chamber via an air circulation fan to cool off the at least one source of UV light and/or dry off the at least one ultrasound probe positioned in the disinfection chamber.

24. The method of claim 16, further comprising the step of rinsing the at least one ultrasound probe positioned in the disinfection chamber with a disinfection solution prior to the step of supplying UV light to the chamber via the at least one source of UV light.

25. The method of claim 16, wherein the at least one source of UV light comprises two or more UV lamps and wherein the method further comprises the step of rotating the lamps around the at least one ultrasound probe positioned in the disinfection chamber.
